(19) United States
(12) Patent Application Publication
(10) Pub. No.: US 2024/0092823 A1
(43) Pub. Date: Mar. 21, 2024

(54) PROCESS FOR THE DE-TRITYLATION OF OLIGONUCLEOTIDES

(71) Applicant: Hoffmann-La Roche Inc., Little Falls, NJ (US)

(72) Inventors: Marek Stanislaw KOMISARSKI, Bottmingen (CH); Pascal MUELLER, Muttenz (CH); Martin OLBRICH, Lörrach (DE)

(73) Assignee: Hoffmann-La Roche Inc., Little Falls, NJ (US)

(21) Appl. No.: 18/449,862

(22) Filed: Aug. 15, 2023

Related U.S. Application Data
(63) Continuation of application No. PCT/EP2022/053556, filed on Feb. 15, 2022.

(30) Foreign Application Priority Data
Feb. 17, 2021 (EP) 21157508.9

(51) Int. Cl. C07H 21/04

(52) U.S. Cl. CPC C07H 21/04

(57) ABSTRACT

The invention relates to a novel process for the production of a linear P-linked oligonucleotide which comprises the removal of the acid labile 5′hydroxy protecting group at the 5′-O oligonucleotide with a detritylation solution comprising acetonitrile. The process allows to produce oligonucleotides with low content of depurination and N−1 impurities.

### CROSS-REFERENCE TO RELATED APPLICATIONS

This application is a Continuation of International Application No. PCT/EP2022/053556, filed Feb. 15, 2022, which claims the benefit of European Patent Application No. 21157508.9 filed Feb. 17, 2021, each of which is incorporated by reference herein in its entirety.

### SEQUENCE LISTING

This application contains a Sequence Listing which has been submitted electronically in XML format and is hereby incorporated by reference in its entirety. Said XML copy, created on Aug. 11, 2023, is named P36710-US-SequenceListing.xml and is 1,718 bytes in size.

The invention relates to a novel process for the production of a linear P-linked oligonucleotide which comprises the removal of the acid labile 5′hydroxy protecting group at the 5′-O oligonucleotide with a detritylation solution comprising a protic acid in a solvent mixture of toluene and acetonitrile.

The oligonucleotide synthesis in principle is a stepwise addition of nucleoside residues to the 5′-terminus of the growing chain until the desired sequence is assembled.

As a rule, each addition is referred to as a synthetic cycle and in principle consists of the chemical reactions

a₁) de-blocking the 5′ protected hydroxyl group on the solid support,

a₂) coupling the first nucleoside as activated phosphoramidite with the free hydroxyl group on the solid support,

a₃) oxidizing or sulfurizing the respective P-linked nucleoside to form the respective phosphodiester (P═O) or the respective phosphorothioate (P═S);

a₄) optionally, capping any unreacted hydroxyl groups on the solid support,

a₅) de-blocking the 5′ hydroxyl group of the first nucleoside attached to the solid support;

a₆) coupling the second nucleoside as activated phosphoramidite to form the respective P—O linked dimer;

a₇) oxidizing or sulfurizing the respective P—O linked dinucleoside to form the respective phosphodiester (P═O) or the respective phosphorothioate (P═S);

a₈) optionally, capping any unreacted 5′ hydroxyl groups;

a₉) repeating the previous steps as to as until the desired sequence is assembled.

The reaction sequence may alternatively start with de-blocking of the 5′ protected hydroxyl group of the nucleoside which is preloaded on the solid support. The subsequent steps follow the sequence as outline above.

Finally, the assembled oligonucleotide is cleaved from the solid support and subsequent downstream processing and purification methods provide the desired pure oligonucleotide.

The de-blocking of the 5′ protected hydroxyl group i.e. the removal of of the acid labile 5′hydroxy protecting group at the 5′-O oligonucleotide is a key repetitive process step in the oligonucleotide synthesis as in each cycle it prepares the so far assembled oligonucleotide for the coupling with the next nucleoside.

The de-blocking process is standard in principle and is well known in the art.

The U.S. Pat. No. 6,538,128 illustrates the standard procedure and discloses the removal of trityl groups which are the typical 5′hydroxy protecting groups with a protic acid such as dichloro- or trichloroacetic acid in the presence of an arene solvent, usually toluene.

The U.S. Pat. No. 6,538,128 further discusses solvents such as methylene chloride and acetonitrile. It was described that acetonitrile slows down the detritylation rate (example 2, line 18) and table 1.

The reason for the slowing down effect has been discussed in C. H. Paul et al, 3048-3052, Nucleic Acids Research, 1996, Vol. 24, No. 15. It was postulated that acetonitrile forms a complex with the protic acid and in competition with the oligo nucleotide drastically slows the detritylation.

The PCT International Publication WO 2012/059510 discloses methods for reducing the pressure in a solid support column caused by the swelling of the solid support in various solvents used for instance in the detritylation process as part of the oligonucleotide synthesis. It is suggested to wash the solid support with a washing fluid comprising methylene chloride or an arene, like toluene, either prior or after the detritylation reaction, which will reduce the pressure built up in the course of the oligonucleotide synthesis. The washing solution may comprise acetonitrile. The detritylation follows the standard method, i.e. applying an acidic solution containing DCA in toluene. The reduction of unwanted side reductions is not object of this PCT disclosure.

Object of the present invention was to further improve the process for the de-blocking the 5′ protected hydroxyl group, particularly the reduction of unwanted side reactions such as the formation of N−1 impurities and the reduction of depurination.

It was found that the object could be achieved with a process for removal of the acid labile 5′hydroxy protecting group at the 5′-O oligonucleotide which is performed with a protic acid in a solvent mixture of toluene and acetonitrile.

The process of the present invention surprisingly delivers significantly less detritylation related side products due to decreased hydrolysis of the glycosidic bond of purine bases in the presence of acetonitrile while at the same time the kinetics of the detritylation is not influenced negatively.

The following definitions are set forth to illustrate and define the meaning and scope of the various terms used to describe the invention herein.

The term acid labile 5′hydroxy protecting group is defined as a protecting group which is cleavable with the help of a suitable acid and which has a hydrophobic character.

Typical acid labile 5′hydroxy protecting groups are selected from 4,4′-dimethoxytrityl, 4-methoxytrityl, trityl, 9-phenyl-xanthen-9-yl, 9-(p-tolyl)-xanthen-9-yl or from tert-butyldimethylsilyl, preferably from 4,4′-dimethoxytrityl, 4-methoxytrityl or trityl or even more preferably from 4,4′-dimethoxytrityl.

The term oligonucleotide as used herein is defined as it is generally understood by the skilled person as a molecule comprising two or more covalently linked nucleotides.

For use as a therapeutically valuable oligonucleotide, oligonucleotides are typically synthesized as 10 to 40 nucleotides, preferably 10 to 25 nucleotides in length.

The oligonucleotides may consist of optionally modified DNA, RNA or LNA nucleoside monomers or combinations thereof.

The LNA nucleoside monomers are modified nucleosides which comprise a linker group or a bridge between C2′ and C4′ of the ribose sugar ring of a nucleotide. These nucleosides are also termed bridged nucleic acid or bicyclic nucleic acid (BNA) in the literature.

Optionally modified as used herein refers to nucleosides modified as compared to the equivalent DNA, RNA or LNA nucleoside by the introduction of one or more modifications of the sugar moiety or the nucleobase moiety. In a preferred embodiment the modified nucleoside comprises a modified sugar moiety, and may for example comprise one or more 2′ substituted nucleosides and/or one or more LNA nucleosides. The term modified nucleoside may also be used herein interchangeably with the term “nucleoside analogue” or modified “units” or modified “monomers”.

The DNA, RNA or LNA nucleosides are as a rule linked by a phosphodiester (P═O) and/or a phosphorothioate (P═S) internucleoside linkage which covalently couples two nucleosides together.

Accordingly, in some oligonucleotides all internucleoside linkages may consist of a phosphodiester (P═O), in other oligonucleotides all internucleoside linkages may consist of a phosphorothioate (P═S) or in still other oligonucleotides the sequence of internucleoside linkages vary and comprise both phosphodiester (P═O) and phosphorothioate (P═S) internucleoside.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are described with capital letters A, T, G and ^{Me}C (5-methyl cytosine) for LNA nucleoside and with small letters a, t, g, c and ^{Me}c for DNA nucleosides. Modified nucleobases include but are not limited to nucleobases carrying protecting groups such as tert.butylphenoxyacetyl, phenoxyacetyl, benzoyl, acetyl, isobutyryl or dimethylformamidino.

The described principles of the oligonucleotide synthesis are well known in the art (see Wikipedia contributors. “Oligonucleotide synthesis” Wikipedia, The Free Encyclopedia, 19 Jan. 2021. Web. 16 Feb. 2021).

Larger scale oligonucleotide synthesis nowadays is carried automatically using computer controlled synthesizers.

As a rule, oligonucleotide synthesis is a solid-phase synthesis, wherein the oligonucleotide being assembled is covalently bound, via its 3-terminal hydroxy group, to a solid support material and remains attached to it over the entire course of the chain assembly. Suitable supports are the commercial available macroporous polystyrene supports like the Primer support 5G from GE Healthcare or the NittoPhase®HL support from Kinovate, or controlled pore glass supports like the nucleobase pre-loaded support from LGC.

As outlined above the oligonucleotide synthesis in principle is a stepwise addition of nucleoside residues to the 5′-terminus of the growing chain until the desired sequence is assembled as outlined above.

The subsequent cleavage from the resin can be performed with concentrated aqueous ammonia. The protecting groups on the phosphate and the nucleobase are also removed within this cleavage procedure.

As outlined above the process of the present invention relates to a process for the production of a linear P-linked oligonucleotide which comprises the removal of the acid labile 5′hydroxy protecting group at the 5′-O oligonucleotide with a protic acid in a solvent mixture of toluene and acetonitrile.

The protic acid is as a rule selected from acetic acid, chloroacetic acid, dichloroacetic acid or trichloroacetic acid. Preferred protic acid is dichloroacetic acid.

Typical acid labile 5′ hydroxy protecting group can be selected from 4,4′-dimethoxytrityl, 4-methoxytrityl, trityl, 9-phenyl-xanthen-9-yl, 9-(p-tolyl)-xanthen-9-yl or from tert-butyldimethylsilyl, preferably from 4,4′-dimethoxytrityl, 4-methoxytrityl or trityl or more preferably from 4,4′-dimethoxytrityl.

The acetonitrile concentration in the solvent mixture with protic acid in toluene is usually in the range of 0.1% (v) to 70% (v), preferably 10% (v) to 25% (v).

The protic acid concentration in toluene is usually selected in the range of 3% (v) and 20% (v), preferably 7% (v) to 17% (v).

Accordingly, in a preferred embodiment the concentration of dichloroacetic acid in toluene is selected in the range of 3% (v) and 20% (v), preferably 7% (v) to 17% (v).

The flow rate of the final detritylation solution after mixing acetonitrile with the protic acid in toluene, is usually in the range of 0.1 CV/min to 2.0 CV/min, preferably 0.3 CV/min to 1.7 CV/min.

As a typical example, the protic acid solution in toluene is mixed in-line together with acetonitrile using the standard oligonucleotide synthesis instrument by setting the pump rates accordingly to the respective ratio of the final flow rate e.g. 0.85 CV/min protic acid solution in toluene and 0.15 CV/min acetonitrile for a total flow rate of 1.0 CV/min at a ratio of 85% protic acid solution in toluene and 15% acetonitrile.

With the conditions of the process of the present invention a level of depurination, expressed as “sum of depurination related impurities” of below 8%, of below 7.0%, of below 6.0%, more preferably below 5.0% can be reached.

Furthermore, with the conditions of the process of the present invention a level of N−1 impurities, expressed as “sum of N−1 impurities” of below 3.0%, of below 2.5%, of below 2.0%, more preferably of below 1.5% can be reached.

This levels can be achieved and measured at the crude oligonucleotide stage, i.e. for the oligonucleotide obtained after cleavage and deprotection and before any downstream processing like purification or ultrafiltration is applied.

In some embodiments, the resin bound oligonucleotide intermediate is either washed with a mixture of acetonitrile and toluene before the detritylation step.

In other embodiments, the resin bound oligonucleotide intermediate is washed with a mixture of acetonitrile and toluene after the detritylation step.

In a preferred embodiment, the resin bound oligonucleotide intermediate is washed with a mixture of acetonitrile and toluene before and after the detritylation step.

The acetonitrile concentration in the solvent mixture with toluene for the washing is usually in the range of 0.1% (v) and 70% (v), preferably 10% (v) to 25% (v).

By way of illustration the oligonucleotide can be selected from:

T*T*A*c*A*c*t*t*a*a*t*t*a*t*a*c*t*T*^{Me}C*^{Me}C

Wherein * stands for phosphorthioate bridges; A, T and ^{Me}C (5-methyl cytosine) are LNA nucleoside monomers and a,t,c are DNA nucleoside monomers.

The compounds disclosed herein have the following nucleobase sequences SEQ ID No. 1: ttacacttaattatacttcc

### EXAMPLES

### Abbreviations

Ac₂O=acetic acid anhydride
BTT=5-benzylthiotetrazol
Bz=benzyl
DCA=dichloroacetic acid
DEA=diethylamine
DNA=2′-deoxyribonuleotide
DMT=4,4′-dimethoxytrityl
CV=column volume
LNA=2′-O—CH₂-4′-bridged ribonucleotide
MeCN=acetonitrile
NA=not applicable
NMI=N-methyl imidazole
PhMe=Toluene

### Example 1

Synthesis of T*T*A*c*A*c*t*t*a*a*t*t*a*t*a*c*t*T*^{Me}C*^{Me}C

Wherein * stands for phosphorthioate bridges; A, T and ^{Me}C (5-methyl cytosine) are LNA nucleoside monomers and a,t,c are DNA nucleoside monomers.

The title compound was produced by standard phosphoramidite chemistry on solid phase at a scale of 2.65 mmol using an AKTA Oligopilot 100 and Primer Support Unylinker (NittoPhase LH Unylinker 330).

The following phosphoramidites have been used in each cycle:

| Cycle | P-amidite | Cycle | P-amidite |
|---|---|---|---|
| 1 | lE(Bz) | 11 | dA(Bz) |
| 2 | lE(Bz) | 12 | dA(Bz) |
| 3 | lT | 13 | dT |
| 4 | dT | 14 | dT |
| 5 | dC(Bz) | 15 | dC(Bz) |
| 6 | dA(Bz) | 16 | lA(Bz) |
| 7 | dT | 17 | dC(Bz) |
| 8 | dA(Bz) | 18 | lA(Bz) |
| 9 | dT | 19 | lT |
| 10 | dT | 20 | lT |

In general 1.5 equiv. of the phosphoramidites were employed. All reagents were used as received from commercially available sources and reagent solutions at the appropriate concentration were prepared (see details below). Cleavage and deprotection was achieved using ammonium hydroxide to give the crude oligonucleotide.

Standard Reagent Solutions

| Deblock | See examples |
|---|---|
| Phosphoramidites | 0.2M in acetonitrile |
| Activator | 0.3M BTT in acetonitrile |
| Thiolation | 0.1M Xanthanhydride in pyridine/acetonitrile |
| | (3/7 v/v) |
| Cap A | NMI/acetonitrile 20/80 (v/v) |
| Cap B | Acetic anhydride/2,6-lutidine/acetonitrile 20/30/50 |
| | (v/v/v) |
| Amine wash | 20% diethylamine in acetonitrile (v/v) |
| Cleavage and | 28-32% aqueous ammonium hydroxide at 55° C. |
| Deprotection | |

The crude solution from the cleavage & deprotection step was concentrated in vacuo to remove excess ammonia. The concentrated solution was lyophilized to provide the crude oligonucleotide as a solid. The pale yellow solid was sampled and submitted to LC-UV-MS analysis. Impurities were grouped according to their assigned structure. The sum of all N−1 impurities and the sum of all depurination related impurities were used for the analysis of the process parameters.

### Example 2

### Detritylation Examples

Detritylation was performed using various solutions of dichloroacetic acid in toluene and acetonitrile as outlined in the table below. The mixtures were delivered to the column by in-line mixing of dichloroacetic acid in toluene with acetonitrile in the ratio provided in the table.

| | Mixing | Concen- | | | | Sum of |
|---|---|---|---|---|---|---|
| | ratio of | tration | | | | De- |
| | Acetonitrile | of | | Sum of | | purination |
| | with DCA | DCA in | | N-1 | MS | related |
| Ex- | in toluene | Toluene | Flow | impurities | Purity | impurities |
| ample | [vol %] | [vol %] | [CV/min] | [%] | [%] | [%] |
| 2a) | 15.0 | 8.82 | 1.5 | 2.50 | 81.53 | 4.61 |
| 2b) | 20.0 | 15.63 | 1.5 | 1.19 | 83.73 | 4.76 |
| 2c) | 17.5 | 12.12 | 1.0 | 1.44 | 81.95 | 5.42 |
| 2d) | 17.5 | 12.12 | 1.0 | 1.31 | 81.77 | 5.36 |
| 2e) | 20.0 | 9.38 | 0.5 | 2.06 | 79.53 | 6.12 |
| 2f) | 20.0 | 15.63 | 0.5 | 1.39 | 78.61 | 7.52 |
| 2g) | 15.0 | 8.82 | 0.5 | 1.34 | 80.37 | 6.63 |
| 2h) | 17.5 | 12.12 | 1.0 | 1.93 | 81.05 | 4.91 |
| 2i) | 17.5 | 12.12 | 1.0 | 1.05 | 82.53 | 5.02 |
| 2j) | 15.0 | 14.71 | 1.5 | 0.77 | 82.09 | 5.04 |
| 2k) | 15.0 | 10.0 | 1.0 | 1.39 | 85.92 | 2.59 |
| 2l) | 10.0 | 10.0 | 1.6 | 1.78 | 80.60 | 5.58 |
| 2m) | 0 | 10.0 | 1.0 | 1.61 | 77.07 | 9.90 |
| 2n) | 0 | 8.5 | 1.0 | 2.62 | 76.22 | 8.38 |
| 2o) | 0 | 3.0 | 2.0 | 2.01 | 73.25 | 8.69 |

Examples 2b), 2c), 2d), 2h, 2i), 2j) and 2k) are regarded as preferred. Examples 2b) and 2k) are most preferred.

Examples 2m) to 2o) are comparison examples.

1. A process for the production of a linear P-linked oligonucleotide comprising contacting a 5′-O oligonucleotide having an acid labile 5′ hydroxy protecting group with a detritylation solution comprising a protic acid in a solvent mixture of toluene and acetonitrile to remove the acid labile 5′ hydroxy protecting group.

2. The process of claim 1, wherein the protic acid is selected from acetic acid, chloroacetic acid, dichloroacetic acid and trichloroacetic acid.

3. The process of claim 2, wherein the protic acid is dichloroacetic acid.

4. The process of claim 1, wherein the acid labile 5′ hydroxy protecting group is selected from 4,4′-dimethoxytrityl, 4-methoxytrityl, trityl, 9-phenyl-xanthen-9-yl, 9-(p-tolyl)-xanthen-9-yl and tert-butyldimethylsilyl.

5. The process of claim 4, wherein, the acid labile 5′ hydroxy protecting group is 4,4′-dimethoxytrityl, 4-methoxytrityl or trityl.

6. The process of claim 1, wherein the acetonitrile concentration in the solvent mixture with toluene is in the range of 0.1% (v) to 70% (v).

7. The process of claim 14, wherein the concentration of the protic acid in toluene is in the range of 3% (v) and 20% (v).

8. The process of claim 1, wherein the removal of the acid labile 5′_hydroxy protecting group takes place the detritylation solution is added at a flow rate of 0.1 CV/min to 2.0 CV/min.

9. The process of claim 1, further comprising washing the 5′-O oligonucleotide with a mixture of acetonitrile and toluene before or after the removal of the acid labile 5′_hydroxy protecting group at the 5′-O oligonucleotide.

10. The process of claim 1, wherein the sum of depurination related impurities (the level of depurination) of the linear P-linked oligonucleotide is below 8.0%.

11. The process of claim 1, wherein the sum of N−1 impurities (the level of N−1 impurities) of the linear P-linked oligonucleotide is below 3.0%.

12. The process of claim 10, wherein the level of depurination is measured in the resulting linear P-linked oligonucleotide after cleavage and deprotection and before any downstream processing is applied.

13. The process of claim 6, wherein the acetonitrile concentration in the solvent mixture with toluene is in the range of 10% (v) to 25% (v).

14. The process of claim 1, further comprising mixing the protic acid in toluene with acetonitrile to form the detritylation solution.

15. The process of claim 7, wherein the concentration of the protic acid in the solvent mixture is in the range of 7% (v) to 17% (v).

16. The process of claim 11, wherein the level of N−1 impurities is measured in the resulting linear P-linked oligonucleotide after cleavage and deprotection and before any downstream processing is applied.


SEQUENCE LISTING (1 sequence)

SEQ ID NO: 1 - DNA, 20 bp, synthetic construct
  source 1..20: mol_type = other DNA
ttacacttaattatacttcc